(19) United States
(12) Patent Application Publication
(10) Pub. No.: US 2006/0247570 A1
(43) Pub. Date: Nov. 2, 2006

(54) Cardiac support cannula device and method

(76) Inventor: James L. Pokorney, Northfield, MN (US)

Correspondence Address: James L. Pokorney, 303 Washington Street, Northfield, MN, 55057, US

(21) Appl. No.: 11/335,398

(22) Filed: Jan. 18, 2006

Related U.S. Application Data
(60) Provisional application No. 60/645,177, filed on Jan. 19, 2005.

(51) Int. Cl. A61F 2/24

(52) U.S. Cl. 604/9; 623/2.11

(57) ABSTRACT

This invention describes apical access methods and devices that enable repair, modification, removal, and/or replacement of a defective heart valve while allowing the beating heart to deliver blood flow through the defective valve annulus for an extended period of time in volumes sufficient to sustain life. The invention is comprised of a flow housing having a blood inflow opening, a blood outflow opening, and a one-way valve disposed within the flow housing between the inflow and outflow openings.

This application claims priority from provisional patent application U.S. Ser. No. 60/645,177 filed 2005 Jan. 19.

### BACKGROUND

1. Field of Invention

The present invention relates generally to methods and systems for cardiovascular surgery. More particularly, the invention relates to less invasive apical access methods and devices that repair, modify, remove, and/or replace a defective heart valve while simultaneously allow the heart to continue delivering blood flow through the defective valve annulus sufficient to sustain life.

2. Clinical Need

Various surgical techniques may be used to repair a diseased or damaged heart valve, such as annuloplasty (contracting the valve annulus), quadrangular resection (narrowing the valve leaflets), commissurotomy (cutting the valve commissures to separate the valve leaflets), or decalcification of valve and annulus tissue. Alternatively, the diseased heart valve may be replaced or pushed aside by a prosthetic valve.

A number of different strategies have been used to repair or replace a defective heart valve such as open heart valve repair, indirect percutaneous valve repair, and direct apical access valve repair.

Open-heart valve repair or replacement surgery is a long and tedious procedure and involves a gross thoracotomy, usually in the form of a median sternotomy. A large opening into the thoracic cavity is created through which the surgeon directly visualizes and operates upon the exposed heart valve. The patient must be placed on cardiopulmonary bypass for the duration of the surgery.

Open-chest valve replacement surgery has the benefit of permitting the careful implantation of the replacement valve with the heart at complete rest. This is possible because the external cardiopulmonary bypass system circulates and oxygenates the body's blood. Because the heart is not actively beating, the surgeon can spend a considerable amount of time ensuring that the valve is implanted into the correct position and is firmly secured to the heart wall. This method, however, is highly invasive and often results in significant trauma, risk of complications, as well as extended hospitalization and painful recovery period for the patient. It is known by those knowledgeable in the art that a significant source of complications can be attributed to the use of the external cardiopulmonary bypass blood circuit.

A less invasive percutaneous valve replacement method has emerged as an alternative to open-chest surgery. U.S. Pat. No. 6,908,481 is representative of this method. Unlike direct open-heart procedures, this procedure is indirect in that it involves accessing the heart valve through employing an intravascular catheter inserted percutaneously into an outlying artery. Because the minimally invasive approach requires only a small incision and because the heart and lungs are not stopped, it allows for a faster recovery for the patient with less pain and trauma. This, in turn, reduces the medical costs and the overall disruption to the life of the patient.

The use of a percutaneous approach, however, introduces new complexities compared to open-heart surgery. One inherent difficulty in the minimally invasive percutaneous approach is the limited space that is available within the vasculature. Unlike open heart surgery, minimally invasive heart surgery offers a surgical field that is only as large as the diameter of a blood vessel. Consequently, the introduction of tools and prosthetic devices becomes a great deal more complicated. A second inherent difficulty in the beating heart procedure is that the implant must be positioned and permanently affixed to the heart in a relatively short time period to ensure blood flow out of the heart is not significantly impeded. Unlike open-heart valve repair that can be performed over a time period of approximately one half hour or so, a percutaneous repair on a beating heart needs to be done in less than one minute to maintain adequate blood flow to the body.

A more direct apical access valve replacement/repair method has emerged as a less invasive alternative to indirect percutaneous repair. US Patent Application 20050240200 is representative of this method. Unlike percutaneous procedures, this procedure allows more direct access to the beating heart through a surgical incision through the apex of a beating heart. And also unlike percutaneous methods and related implants, the apical approach allows for larger and less complicated delivery methods and implants. Therefore, in many ways, this approach is superior to percutaneous approaches. Unfortunately, the apical installation methods known in the prior art also require a short implant time similar to percutaneous approaches. Considering the implant needs to be securely sealed to the heart annulus and will open and close over 3 million times per year for as many as twenty years, a less than optimum installation due to a limited time period is a significant shortcoming of both the percutaneous and apical procedures.

Accordingly, while apical access heart valve surgery has the potential to produce beneficial results equal to or superior to open-chest methods or percutaneous methods, the requirement that the procedure needs to be done quickly because the valve implantation tools occlude heart outflow tract limits the procedure's potential for high quality clinical success. Therefore, what is needed are apical access methods and devices that enable repair, modification, removal, and/or replacement of a defective heart valve while allowing the beating heart to deliver uninterrupted blood flow through the defective valve annulus for an hour or so in volumes sufficient to sustain life.

### OBJECTS AND ADVANTAGES

The primary object of the present invention is not focused on any specific therapeutic mechanisms or implants located near or on the outside surface of an apically inserted device, but instead, the primary object of the present invention is focused on the mechanisms and methods required to extend the time these therapies can be performed by providing a means for controlled one-way blood flow through the inside of the device while therapy is performed near or on the outside of the device.

Specifically, the invention has the following advantages:
The invention enables therapeutic devices to act on an immobilized heart valve over a considerable length of time while allowing controlled life sustaining blood flow through the therapeutic device.
The invention requires no energy source other than the human heart to control blood flow through the annulus formed by an incompetent heart valve.
The invention can be inserted through the apex of a beating heart.
Therapeutic mechanisms or implants can be located near or on the outside surface of the invention.

These and other objects and advantages of this invention are achieved by a mechanism comprises a flow housing or internal lumen having at least one blood inflow opening, at least one blood outflow opening, and a one-way valve disposed within the flow housing between the inflow and outflow openings.

The above mentioned objects and advantages of this invention will become apparent from the following description taken in connection with the accompanying drawings, wherein is set forth by way of illustration and example, preferred embodiments of this invention.

### DESCRIPTION OF DRAWING FIGURES

In the drawings, closely related figures have the same number but different alphabetic prefixes.

FIG. 1 shows a perspective view of one embodiment of the invention.

FIG. 2A shows a side view end view of one embodiment of the invention.

FIG. 2B shows a cross section of the end view shown in FIG. 2A.

FIG. 3A shows a side view and end view of one embodiment of the invention with balloons not inflated.

FIG. 3B shows a side view and end view of one embodiment of the invention with balloons inflated.

FIG. 3C shows a cross section of the end view shown in FIG. 3A.

FIG. 4A shows a side view and end view of one embodiment of the invention with an associated implant with balloons not inflated.

FIG. 4B shows a side view and end view of one embodiment of the invention with an associated implant with balloons inflated.

FIGS. 5A-F are a series of combination side views and cross-sectional views of one embodiment of the invention being used in a beating heart to implant a heart valve.

### DEFINITIONS

The terms “proximal” and “distal,” when used herein in relation to the invention or the procedure described in the invention respectively refer to directions closer to and farther away from the surgeon performing the procedure.

General Summary of Invention

When performing direct apical access valve repair or replacement procedures such as but not limited to annuloplasty (expanding the valve annulus), quadrangular resection (narrowing the valve leaflets), commissurotomy (cutting the valve commissures to separate the valve leaflets), decalcification of valve and annulus tissue, or replacement with a prosthetic valve, it is necessary to insert a device through the apex of the heart into any of the various chambers of the heart and major vessels connected to the heart. The apically deployed device can be of many sizes and shapes, including generally flexible polymer tubes, wire-reinforced polymer tubes, and even rigid metal tubes. The particular valve therapy being performed, examples of which are provided above, generally requires a mechanism at or near the outside surface of the device. These mechanisms generally need to fill the annular space at the site of the diseased valve to affect therapy, thereby significantly occluding flow through the valve during the time of therapy.

The present invention is not focused on any of these specific therapeutic mechanisms or implants located near or on the outside surface of the apically inserted device, but instead is focused on the mechanisms and methods required to extend the time these therapies can be performed by providing means for controlled one-way blood flow through the inside of the device while therapy is performed near or on the outside of the device.

The invention described herein enables therapeutic devices to act on an immobilized heart valve over a considerable length of time while allowing controlled life sustaining blood flow through the therapeutic device.

The invention described herein is not intended to work with only one such possible therapeutic device intended to be placed in the annulus of a beating heart, but is intended to make practical many possible therapies that require prolonged access to an isolated and incompetent heart valve.

In more detail, the present invention describes mechanisms and associated methods for allowing temporary flow control of a patient's heart. A preferred mechanism comprises a flow housing or internal lumen having at least one blood inflow opening, at least one blood outflow opening, and a one-way valve disposed within the flow housing between the inflow and outflow openings. An associated preferred method for allowing temporary flow control of a patient's blood through the aortic valve requires making an incision in the heart adjacent the left ventricles and then inserting the flow housing within the incision such that the inflow opening is located within the ventricle and the outflow opening is located within the aorta. A second preferred method for allowing temporary flow control of a patient's blood through the mitral valve requires making a similar incision in the heart adjacent the left ventricle and then inserting the flow housing within the incision such that the outflow opening is located within the ventricle and the inflow opening is located within the atrium or the pulmonary vein. To those knowledgeable in the art, it is obvious that similar techniques could be employed on the right side of the heart to control blood flow through the pulmonary or tricuspid valve while therapy is being performed on the valve or associated valve annulus.

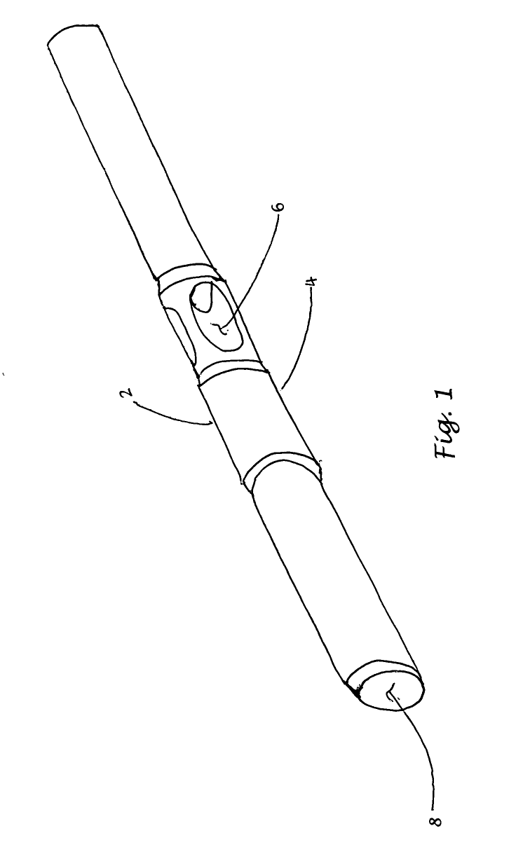

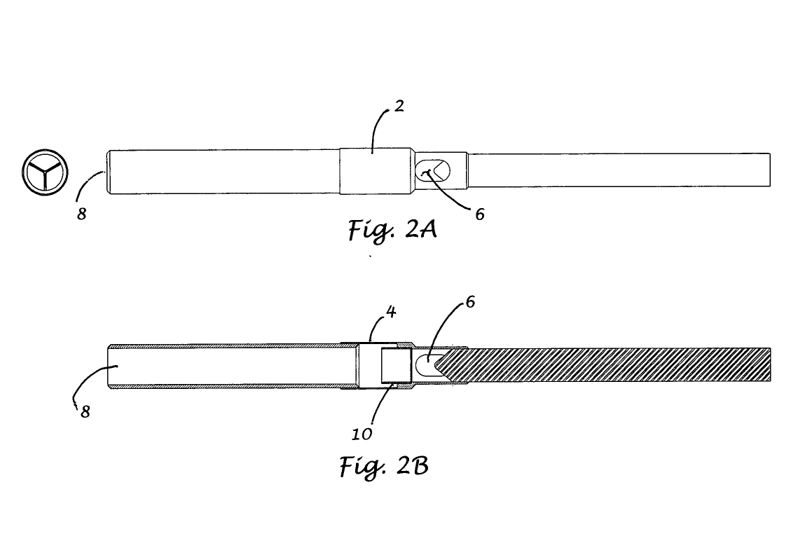

The invention, independent of the specific therapy integrated into the device, is called a Cardiac Support Cannula. One simple embodiment of the invention is shown in FIG. 1. and FIGS. 2A and 2B. In this embodiment is shown a Cardiac Support Cannula 2 that comprises a flow housing or internal lumen 4 having at least one blood inflow opening 6, at least one blood outflow opening 8, and a one-way valve 10 disposed within the flow housing between the inflow and outflow openings.

In another preferred embodiment described in detail below, a stent balloon is included in the device to deliver a balloon expandable valve prosthesis similar to the general class of implants described in U.S. Pat. No. 6,908,481. Also included as an option in the following preferred embodiment is a second balloon located on the exterior surface proximal to the stent balloon. It is used to help isolate the inflow openings from the outflow openings. To those knowledgeable in the art, many other therapeutic mechanisms such as cutters, lasers, thermal elements, ultrasound elements, and other, yet to be invented therapeutic mechanisms could be located at or the near the external surfaces of the Cardiac Support Cannula. These mechanisms do not effect or alter the function of the blood flow control invention described herein.

Description of Invention Structure

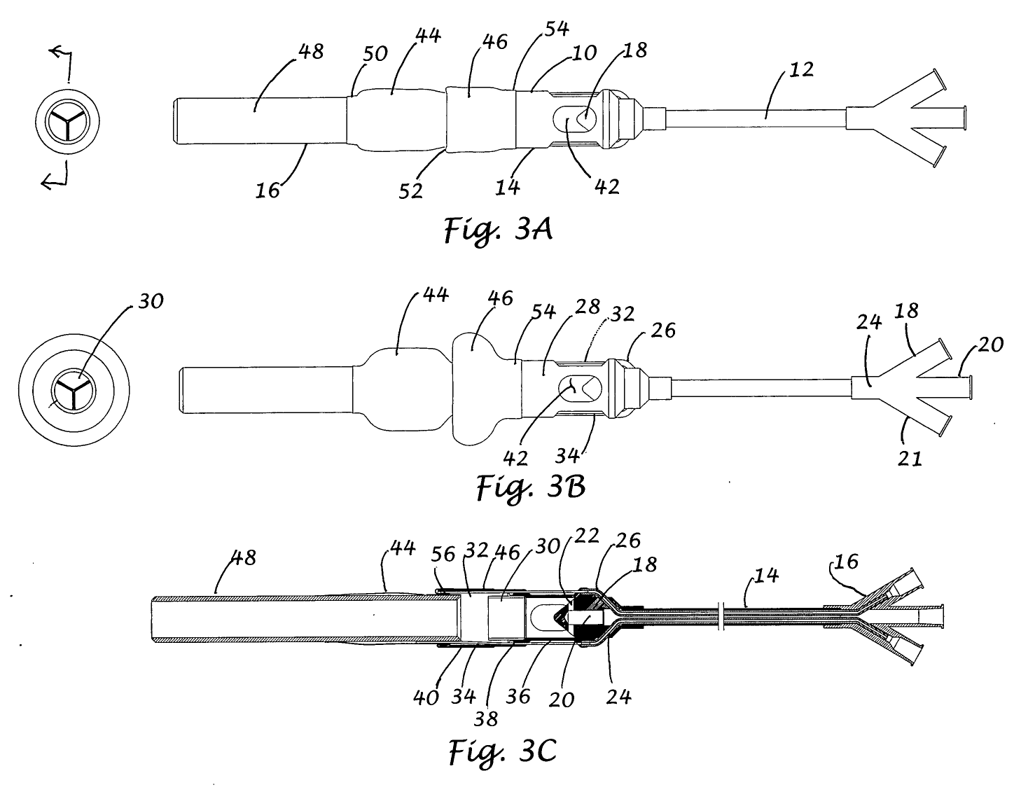

In a preferred embodiment as shown in FIGS. 3A-3C, the Cardiac Support Cannula 10 comprises three main elements: a Transfer Shaft 12, a Flow Housing 14, and an Outflow Cannula 16.

Transfer Shaft

The Transfer Shaft 12 is the most proximal element of the device. The purpose of the Transfer Shaft is to provide user control and orientation of the other, more distal elements of the device and to allow fluid communication with the more distal elements of the device or the heart itself.

The Transfer Shaft 12 comprises a Tubular Member 14 with a Port Connector 16 attached to the proximal end and a Flow Diverter 18 attached to the distal end.

The Port Connector 16 is composed of polycarbonate plastic or some other suitable biocompatible material. The Port Connector 16 has three proximal ports identified as the Annulus Balloon Port 18, the Infusion Port 20, and the Stent Balloon Port 21. These ports are in common communication with one Distal Port 24. The three proximal ports terminate in female luer connectors, a type of standard medical device known to those skilled in the art. The Distal Port 24 is sized to closely engage over the Tubular Member 14.

The Tubular Member 14 is a cylindrical tube composed of stainless steel or another similar biocompatible material. The length of the Tubular Member is such that when the more distal elements are positioned near a selected heart valve inside the heart, the Port Connector 16 is positioned conveniently outside of the heart. Attached to the distal end of the Tubular Member 14 is the Flow Diverter 18.

The Flow Diverter 18 is composed of stainless steel or some other similar blood compatible material. The distal end of this generally cylindrical component is tapered to form a conical shape with a bluntly pointed tip. At the proximal end of the Flow Diverter 18 is an Internal Plenum or hole 20 with a diameter sized to fit the Tubular Member 14. Emanating from the Internal Plenum 20 are five holes that communicate with the outside surface of the Flow Diverter 18. Three of these holes, identified as Infusion Holes 22, exit the Internal Plenum on the distal conical surface of the Flow Diverter. The two other holes, identified as Pass Thru Holes 24, exit onto the major cylindrical surface of the Flow Director 18. Flexible Tubings 26 are inserted into the Pass Thu Holes 24 and lay within the Internal Plenum 20 continuing through the Tubular Member 14 and terminating at the Annulus Balloon Port 18 and the Stent Balloon Port 21. Termination is made by applying an adhesive to form a fluid tight seal between the tubings and the Port Connector 16.

The major outside diameter of the Flow Director 18 is sized to fit into and be attached to the proximal end of the Flow Housing 14.

Flow Housing

In use, the Flow Housing 14 element of the device is inserted into the annulus of a heart valve in a beating heart. Because the native heart valve is made incompetent by the device, the purpose of the Flow Housing 14 is to allow controlled one-way blood flow within the device while preventing blood flow past the outside of the device.

In the preferred embodiment, the Flow Housing 14 is composed of two major components: a Sinus Housing 28 and a Prosthetic Valve 30 contained with the Sinus Housing 28. Also included in the Flow Housing 14 are two Flow Tubes, identified as an Annuls Flow Tube 32 and a Stent Flow Tube 34.

The Sinus Housing 28 is a thin walled, generally cylindrically shaped component open on both ends. The internal hole in the component can be described as having three stepped diameters identified as a Flow Surface 36, a Valve Surface 38, and a Sinus Surface 40. Near the proximal end of the Sinus Housing 28 are located three Blood Inlets Holes 42 spaced evenly about the central axis of the Sinus Housing 28.

Located within the Sinus Housing 28 is a Prosthetic Valve 30 composed of polyurethane or a similar blood compatible material. The Prosthetic Valve 30 is oriented to allow flow through the Sinus Housing 28 from the proximal end to the distal end and prevents flow from the distal end to the proximal end. It is affixed to the Valve Surface 38 of the Sinus Housing 28 using an adhesive. The internal diameter of the Prosthetic Valve 30 is generally equivalent to the diameter of the Flow Surface 36 of the Sinus Housing 28 to facilitate smooth, efficient flow.

The Annulus Flow Tube 32 is a thin walled polymer tube in the preferred embodiment. The purpose of the Annulus Flow Tube 32 is to continue the flow path from the Annulus Balloon Port 18 generally to the outside surface of the Outflow Cannula 16 element of the device. It is inserted into proximal and distal holes located in the wall of the Sinus Housing 28.

The Stent Flow Tube 34 is a thin walled polymer tube in the preferred embodiment. The purpose of the Stent Flow Tube 34 is to continue the flow path from the Stent Balloon Port 21 generally to the outside surface of the Outflow Cannula 16 element of the device. Like the Annulus Flow Tube, it is inserted into proximal and distal holes located in the wall of the Sinus Housing 28.

The flow tubes are located about the circumference such that neither of the tubes lay over any of the Blood Inlet Holes 42.

Outflow Cannula

The Outflow Cannula 16 is the most distal element of the device. The purpose of the Outflow Cannula 16 is to deliver blood beyond the annulus of the heart and to provide a surface to mount the Stent Balloon Sleeve 44 and the distal end of the Annulus Balloon Sleeve 46.

In a preferred embodiment, the Outflow Cannula 16 is composed of three major components: an Outflow Tube 48, the Stent Balloon Sleeve 44, and the Annulus Balloon Sleeve 46.

The Outflow Tube 48 is composed of silicone with a stainless steel wire reinforced coil embedded into the wall of the tube. The purpose of the wire coil is to prevent kinking of the Outflow Tube 48 and is a common design used by many knowledgeable in the art of flexible blood cannulae. The internal diameter of the Outflow Tube 48 is about 10 mm in the preferred embodiment.

The Distal End 50 and the Proximal End 52 of the Stent Balloon Sleeve 44 are attached to the outside surface of the Outflow Tube 48 near the proximal en of the tubed. The Stent Balloon Sleeve 44 is composed of a one of potentially many polymers typically used by those knowledgeable in the art of balloon expandable stents and associated balloons. The polymer material may be nylon or polyurethane for example.

The Proximal End 54 of the Annulus Balloon Sleeve 46 is attached to the distal end of the Sinus Housing 28. After it is bonded using adhesive, heat, or some other bonding method, the Outflow Tube 48 is inserted into the Sinus Housing 28 and bonded in place. Then the Annulus Balloon Sleeve is inverted such that the free, unbonded Distal End 56 of the Annulus Balloon Sleeve is bonded to the proximal end of the Outflow Tube 48.

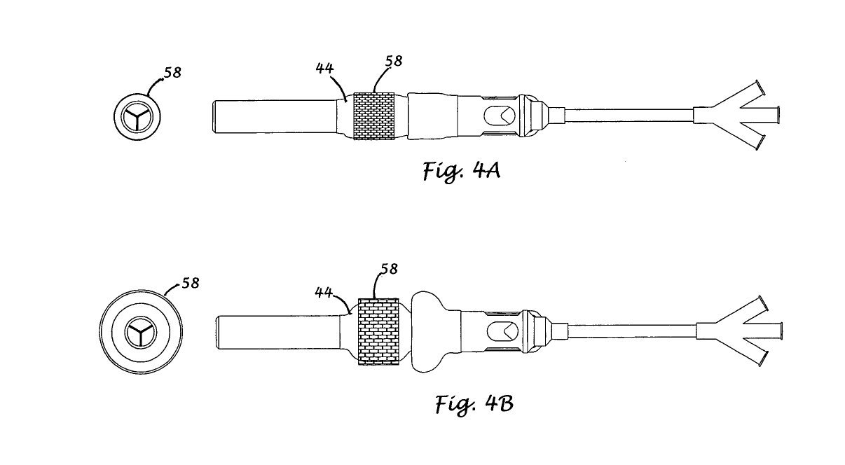

Another embodiment of the invention is shown in FIGS. 4A and 4B. This embodiment is similar to that shown in FIGS. 3A-C with the addition of a balloon expandable heart valve Implant 58 inserted over the Stent Balloon Sleeve 44. The Implant 58 is of similar design and construction known in the art as exemplified by US Patent Application 20050240200. FIG. 4A shows the Implant 58 and Stent Balloon Sleeve 44 and Annulus Balloon 46 all in the uninflated state. FIG. 4B shows the Implant 58 expanded and the Stent Balloon Sleeve 44 expanded by applying fluid pressure to Stent Balloon Port 21 and the Annulus Balloon Sleeve 46 expanded by applying fluid pressure to Annulus Balloon Port 18.

Operation of Cardiac Support Cannula

One application of the invention described is to implant a prosthetic heart valve into a beating heart. This application is representative of how the invention can provide necessary blood flow support while working within the annulus of a beating heart. Other heart leaflet therapy applications such as valvuloplasty, ultrasound decalcification, chemical decalcification, or other applications can all be aided by use of the invention.

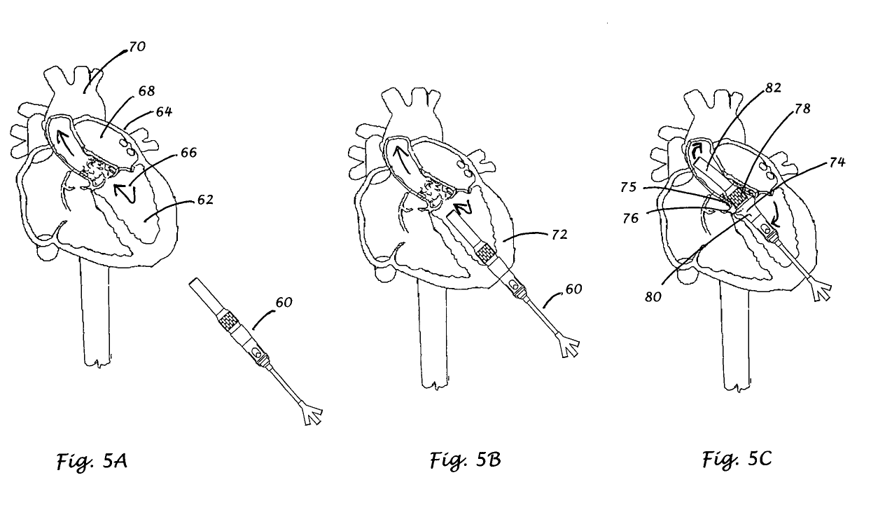

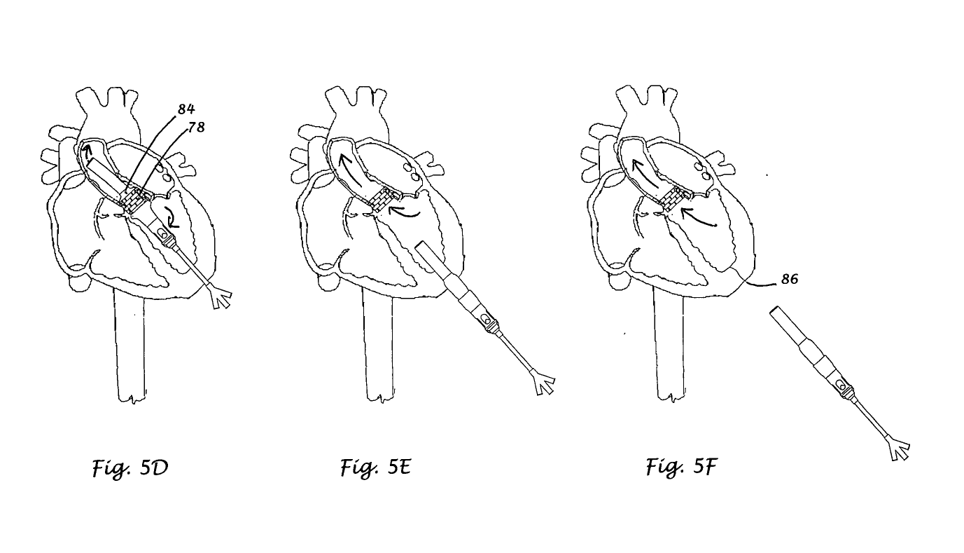

FIG. 5A-F show a representative method of use. FIG. 5A shows the Cardiac Support Cannula 60 ready to be inserted into the Ventricle 62 of the Beating Heart 64 of the patient. Blood Flow Lines 66 are shown to represent blood flow from the Atrium 68 to the aorta 70 via the Ventricle 62. FIG. 5B shows the Cardiac Suppot Cannula 60 being inserted through the Ventricle Wall 72. FIG. 5C shows the Annulus Balloon 74 inflated and placed flush against the Annulus Base 76 to prevent inadvertent, uncontrolled blood flow around the device and demonstrates how the Implant 78 is accurately located within the Annulus 75 by way of the Annulus Balloon 74 being flush against the Annulus Base 76. When in this location, the Beating Heart 64 pumps blood from the 62 Ventricle to the Aorta 70 through the Valve Housing 80 and Outflow Cannula 82 elements of the Cardiac Support Cannula 60. FIG. 5D shows the Stent Balloon Sleeve 84 inflated to deploy the Implant 78. The Cardiac Support Cannula 60 continues to allow sufficient net forward blood flow through the device as shown by Blood Flow Lines 66. FIG. 5E shows both balloons deflated and the Cardiac Support Device 60 removed from the heart. FIG. 5F shows the Implant 78 in place, the Cardiac Support Cannula 60 removed, and the Apical Access Site 86 sutured closed.

Summary, Ramifications, and Scope

Although the description above contains many specifications, these should not be construed as limiting the scope of the invention but as merely providing illustrations of the presently preferred embodiment of this invention. For example:
The transfer shaft diameter could be increased to be approximately equal in diameter to the Flow Housing to allow the Implant to be to be introduced into the heart down the shaft after the Stent Balloon has enlarged the Annulus. In this variation, the Implant need not be expanded within the heart, but could be introduced full size.
Pressure monitoring tubes could be attached to the device at different location to measure blood pressure. This could be useful to assess which heart chamber or vessel the device is located.
A dilator could be inserted through the device in place of the stationary flow diverter. During insertion, the dilator, which would extend beyond the tip of the Outflow Cannula, would assist in traversing the heart. Once in place, the dilator could be retracted until the tip of the dilator is located in about the same location as the tip of the Flow Diverter to help divert flow towards the Outflow Cannula.
A second coaxial tubular device or shroud could be inserted over the Cardiac Support Device to protect the implant or other therapeutic mechanisms during insertion. This Shroud would not impede flow through the Blood Inlet Holes.
   Thus, the scope of the invention should be determined by the appended claims and their legal equivalents rather than by the examples given.

1. A device for allowing temporary flow control of a patient's heart comprising a flow housing having at least one blood inflow opening, at least one blood outflow opening, and a one-way valve disposed within the flow housing between the inflow and outflow openings.

2. A method for allowing temporary flow control of a patient's heart, comprising the steps of:
a) providing a flow housing having at least one blood inflow opening, at least one blood outflow opening, and a one-way valve disposed within the flow housing between the inflow and outflow openings;
b) making an incision in the heart adjacent one of the left and right ventricles;
c) inserting the flow housing within the incision such that within the one ventricle is located an inflow opening and the outflow opening is located within the outflow artery associated with the one ventricle.

3. A method for allowing temporary flow control of a patient's heart, comprising the steps of:
a) providing a flow housing having at least one blood inflow opening, at least one blood outflow opening, and a one-way valve disposed within the flow housing between the inflow and outflow openings;
b) making an incision in the heart adjacent one of the left and right ventricles;
c) inserting the flow housing within the incision such that within the one ventricle is located an outflow opening and the inflow opening is located within the atrium or inflow artery associated with the one ventricle.
